(19) United States
(12) Patent Application Publication
(10) Pub. No.: US 2026/0026690 A1
(43) Pub. Date: Jan. 29, 2026

(54) OPHTHALMIC INSTRUMENT DUST COVER HAVING PROBE LOADING FUNNEL

(71) Applicant: Reichert, Inc., Depew, NY (US)

(72) Inventors: Russell J. BONAVENTURA, Williamsville, NY (US); Alexa J. BARRIBEAU, Buffalo, NY (US)

(21) Appl. No.: 18/784,520

(22) Filed: Jul. 25, 2024

(51) Int. Cl. A61B 3/16

(52) U.S. Cl. CPC A61B 3/165; A61B 2562/247

(57) ABSTRACT

A dust cover for an ophthalmic instrument includes a cover unit and a closure member coupled to the cover unit. The cover unit includes a funnel portion extending along a probe-loading axis and having a mouth and a probe-loading orifice, wherein an area of the probe-loading orifice is reduced relative to an area of the mouth. The closure member is selectively movable relative to the cover unit between an open position in which the mouth of the funnel portion is not fully covered by the closure member and a closed position in which the mouth of the funnel portion is fully covered by the closure member. The dust cover removably mounts onto a measurement head of the ophthalmic instrument, assists a user in loading a new probe into the measurement head, and remains on the measurement head when the ophthalmic instrument is not in use.

### FIELD OF THE DISCLOSURE

The present disclosure relates to ophthalmic instruments which utilize a disposable probe for contacting a cornea of an eye to measure a parameter of the eye. For example, the present disclosure relates to rebound tonometers into which a disposable probe is loaded for measuring intraocular pressure (IOP).

### BACKGROUND OF THE DISCLOSURE

A rebound tonometer is an ophthalmic instrument that propels a movable measurement probe in a controlled manner along a measurement axis toward the cornea of an eye to measure intraocular pressure. During a measurement, the probe contacts the cornea, decelerates at a rate which depends on intraocular pressure, and then rebounds in a direction away from the cornea back toward the instrument housing. The rebound tonometer detects the motion of the measurement probe and determines intraocular pressure based on the detected motion of the probe. For example, the measurement probe may have a magnetized shaft that travels within a coil in the instrument housing. The coil may be energized momentarily to propel the probe toward the cornea by electromagnetic force, and then, after energizing current to the coil is shut off, a current may be induced in the coil by the moving probe to provide a detectable voltage signal representing velocity of the probe as a function of time. Alternatively, two coils may be provided, wherein one coil is used to propel the probe and the moving probe induces current in the other coil to provide a measurement voltage signal. The voltage signal may be recorded and processed to determine a measured IOP value.

Known rebound tonometers are designed for hand-held use by an operator to measure IOP of a test subject's eye. In a common arrangement, the coil or coils are housed within a measurement head of the rebound tonometer that sits atop a handle, and the rebound tonometer is manually positioned close to the test subject's eye for measurement. Prior to measurement, the shaft of the probe must be loaded into an axially elongated tube which extends through an inner diameter of the coil or coils in the measurement head. The shaft of the probe may be a thin wire having a diameter in a range from about 0.293 mm to about 0.320 mm, and the inner diameter of the tube is slightly larger, for example about 0.75 mm. Consequently, loading the probe by gripping the probe with fingers and inserting a rear end of the probe into tube in the measurement head is challenging and often results in a bent or dropped probe that can no longer be safely used for measurement.

One known solution is to provide a separate probe applicator as an accessory with a rebound tonometer to assist a user in loading a probe. The probe applicator has a mounting opening at one end that is sized to mate with a protruding probe base of the measurement head, a mouth at an opposite end, and a converging passageway in the nature of a funnel leading from the mouth toward the mounting opening to guide an inserted probe axially into the tube of the measurement head. Once the probe is loaded using the applicator, the applicator is removed from the probe base of the measurement head to allow for measurement.

The known rebound tonometers and probe applicator have several drawbacks. For example, after the applicator is removed from the probe base of the measurement head and the instrument is used for measurement, the measurement head remains uncovered and is susceptible to dust collection and other contaminants. Unless the instrument is placed into a storage case, these contaminants could be inadvertently spread into a test subject's eye during a future measurement. The requirement to return the instrument to the storage case after each use can be cumbersome to some users, which can cause those users to not use the storage case at all and can increase the likelihood of contamination. Merely mounting the probe applicator onto the probe base after measurement would not protect the measurement head from contamination without the storage case. Even with the storage case, the probe applicator is a separate accessory that must be removed from the measurement head prior to storing and can be easily misplaced.

### SUMMARY OF THE DISCLOSURE

The present disclosure provides a dust cover for an ophthalmic instrument such as a rebound tonometer into which a disposable measurement probe is loaded for taking a measurement. The dust cover protects a measurement head of the instrument while facilitating the loading of a new measurement probe into the measurement head.

The dust cover may comprise a cover unit and a closure member coupled to the cover unit. The cover unit may define an inner enclosure space and a mounting opening communicating with the inner enclosure space. The cover unit may include a funnel portion extending along a probe-loading axis, wherein the funnel portion tapers from a mouth of the funnel portion toward a probe-loading orifice of the funnel portion such that an area of the probe-loading orifice is reduced relative to an area of the mouth. The closure member may be selectively movable relative to the cover unit between an open position in which the mouth of the funnel portion is not fully covered by the closure member and a closed position in which the mouth of the funnel portion is fully covered by the closure member.

In some embodiments, the cover unit further includes a tube portion extending from the funnel portion toward the mounting opening, wherein the tube portion has a probe-loading passageway coaxial with and extending from the probe-loading orifice to terminate at a probe-delivering orifice. The probe-loading orifice may be axially located between the mouth of the funnel portion and the mounting opening of the cover unit, and the probe-delivering orifice may be axially located between the probe-loading orifice and the mounting opening.

In some embodiments, the cover unit includes a pair of slots and an arcuate surface portion between the pair of slots, the mouth of the funnel portion opens through the arcuate surface portion, and the closure member includes an arcuate strip having a pair of opposite ends and an insertion hole through the arcuate strip, wherein the pair of opposite ends of the arcuate strip are received through the pair of slots such that the arcuate strip is substantially flush with the arcuate surface portion and the closure member is slidable relative to the cover unit to align the insertion hole with the mouth of the funnel portion in an open position and to cover the mouth of the funnel portion with the arcuate strip in a closed position. The closure member may further include a tab protruding from the arcuate strip for engagement by a user to move the closure member relative to the cover unit. The tab may protrude from the arcuate strip at a location midway between the pair of opposite ends of the arcuate strip, and the insertion hole may be defined on the arcuate strip at a location between the tab and one end of the pair of opposite ends. The arcuate strip may include a circular arc portion which may measure, for example, at least 180 degrees. In an embodiment of the disclosure, the arcuate strip may be dimensioned such that the arcuate strip is flexed when the pair of opposite ends of the arcuate strip are received through the pair of slots, whereby the arcuate strip is spring-biased to exert a lateral force against respective edges of the pair of slots.

The dust cover of the present disclosure assists a user in loading a new probe into the measurement head of the ophthalmic instrument and remains on the measurement head after a probe is loaded to protect the measurement head and probe prior to a measurement. The dust cover is removed from the measurement head to perform a measurement. After a measurement is performed, the probe may be discarded and the dust cover may again be mounted on the measurement head of the ophthalmic instrument such that the ophthalmic instrument may be safely stored without returning the ophthalmic instrument to a carrying case designed to fully enclose the ophthalmic instrument.

### BRIEF DESCRIPTION OF THE DRAWING VIEWS

The nature and mode of operation of the present invention will now be more fully described in the following detailed description taken with the accompanying drawing figures, in which:

FIG. 1A is an exploded perspective view of an ophthalmic instrument and a dust cover for the ophthalmic instrument wherein the dust cover is formed in accordance with an embodiment of the present disclosure;

FIG. 1B is a perspective view showing the dust cover of FIG. 1A mounted on the ophthalmic instrument;

FIG. 2 is another perspective view of the ophthalmic instrument shown in FIGS. 1A and 1B;

FIG. 3 is a perspective view of the dust cover wherein a closure member of the dust cover is in a closed position;

FIG. 4 is a cross-sectional view of the dust cover wherein the closure member is in the closed position;

FIG. 5 is a perspective view similar to that of FIG. 3, wherein the closure member is in an open position;

FIG. 6 is a cross-sectional view similar to that of FIG. 4, wherein the closure member is in the open position;

FIG. 7A is a perspective view of a cover unit of the dust cover of FIG. 1;

FIG. 7B is another perspective view of the cover unit of the dust cover of FIG. 1;

FIG. 8 is a top plan view of the cover unit shown in FIGS. 7A and 7B;

FIG. 9 is a cross-sectional view of the cover unit taken generally along the line A-A in FIG. 8;

FIG. 10 is a perspective view of the closure member of the dust cover of FIG. 1;

FIG. 11 is a top plan view of the closure member shown in FIG. 10;

FIG. 12 is a cross-sectional view of the closure member taken generally along the line B-B in FIG. 11; and

FIGS. 13A, 13B, and 13C are a series of cross-sectional views of the dust cover on a measurement head of the ophthalmic instrument illustrating loading of a measurement probe into the measurement head of the ophthalmic instrument.

### DETAILED DESCRIPTION OF THE INVENTION

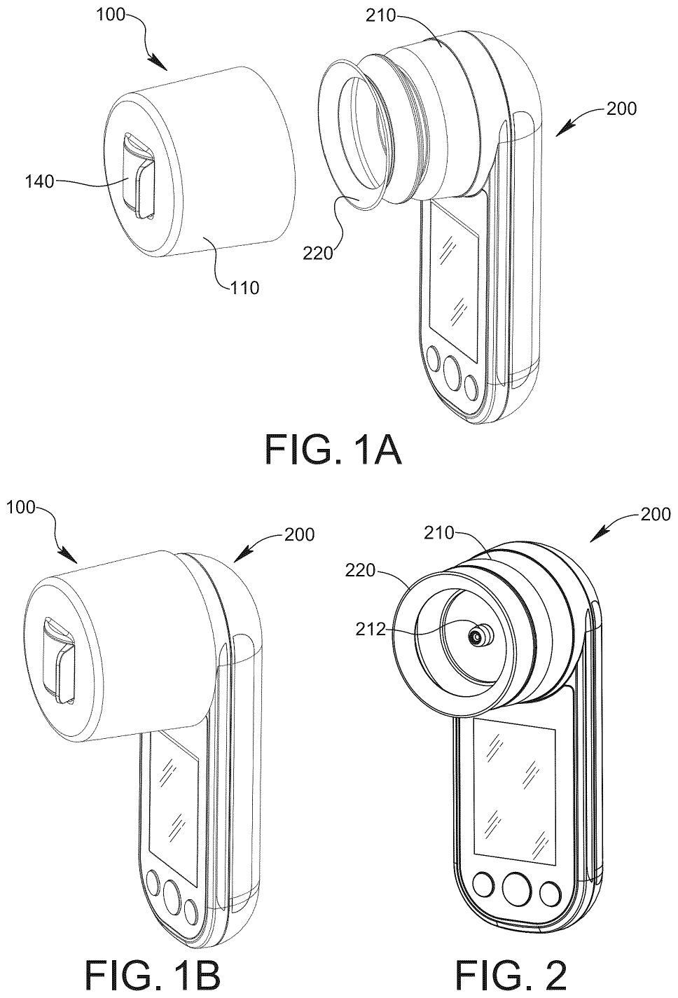

An embodiment of the present disclosure provides a dust cover 100 for an ophthalmic instrument 200, as shown in FIGS. 1A, 1B, and 2. Ophthalmic instrument 200 may be configured to measure one or more parameters of an eye. For example, ophthalmic instrument 200 may be embodied as a rebound tonometer which utilizes a disposable probe to contact the cornea of the eye to measure intraocular pressure (IOP). In another example, ophthalmic instrument 200 may be configured to measure one or more biomechanical properties of a cornea or corneal tissue by contacting the cornea with a disposable probe. The ophthalmic instrument 200 may be designed for hand-held use by user to measure IOP of a test subject's eye or the user's own eye. In the illustrated example shown in FIG. 2, ophthalmic instrument 200 may comprise a measurement head 210 having a probe barrel 212 configured to receive a disposable probe. Ophthalmic instrument 200 may also comprise an eye cup 220 surrounding probe barrel 212. Eye cup 220 may contact the test subject around the eye (e.g., cheekbone, forehead, or other facial area) when positioning measurement head 210 near the eye for measurement. In some embodiments, the eye cup 220 may be made of a deformable, elastomeric material and may be configured similar to a compressible bellows, whereby the eye cup may confirm to a test subject's face around an eye of the test subject while permitting position adjustment of measurement head 210 relative to the enclosed eye in an X direction (left-right), a Y direction (up-down), and a Z direction (toward-away from eye). In some embodiments, eye cup 220 may be removably mounted on measurement head 210. In other embodiments, eye cup 220 may be provided as a permanent part of measurement head 210.

Dust cover 100 may be removably coupled to ophthalmic instrument 200. For example, dust cover 100 may be dimensioned to be removably mounted on measurement head 210 to surround and frictionally engage measurement head 210, with or without eye cup 220 coupled to measurement head 210. In some embodiments, the dust cover 100 may be dimensioned to be removably mounted on probe barrel 212.

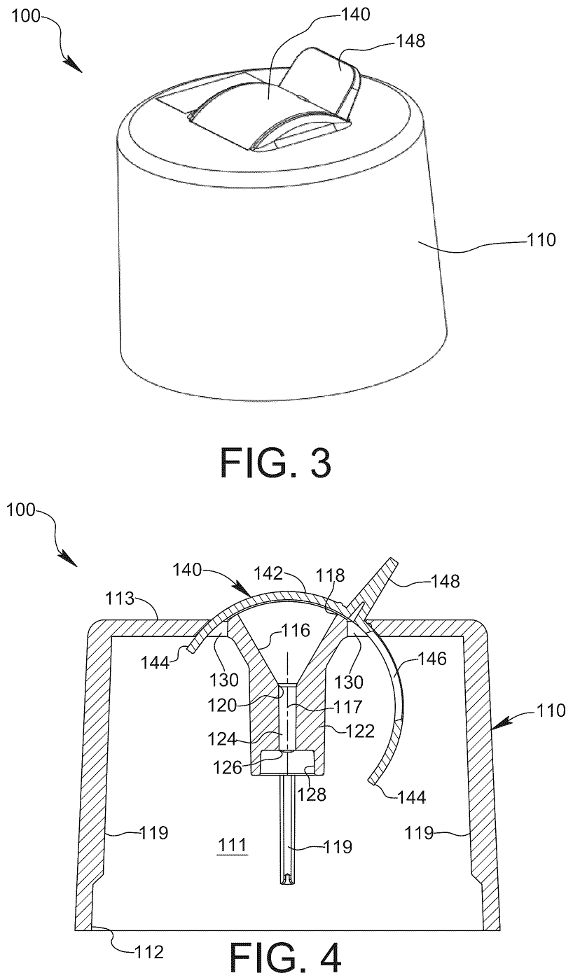

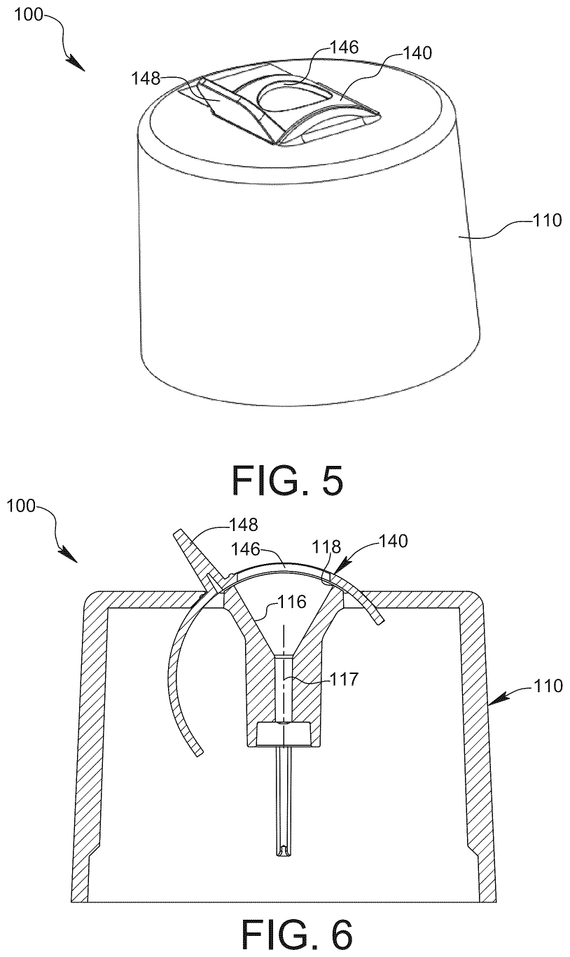

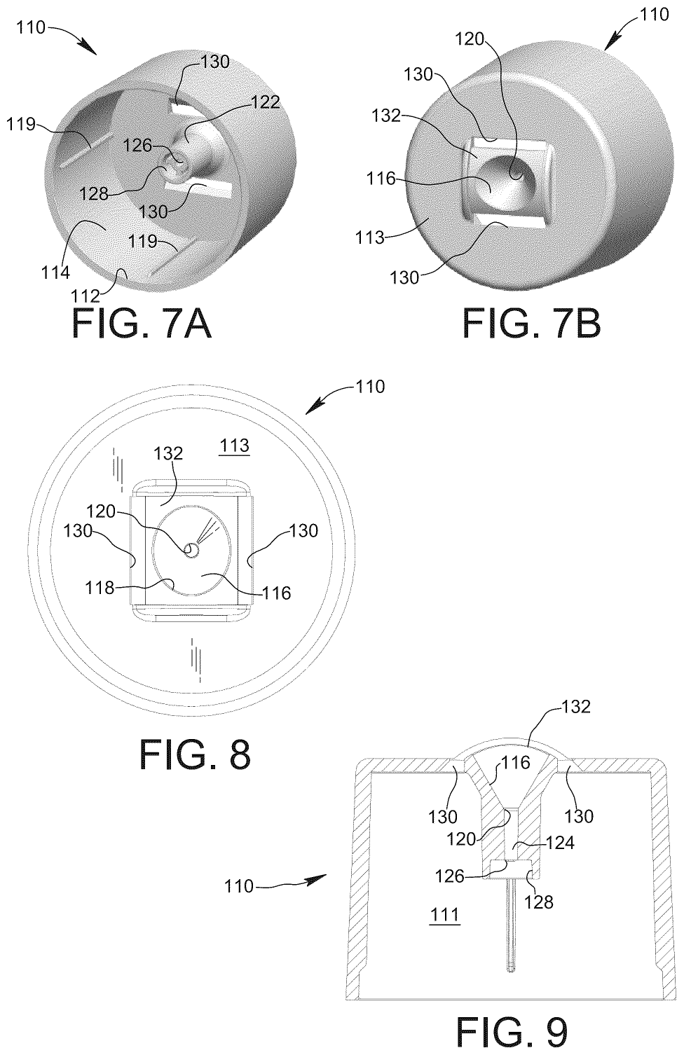

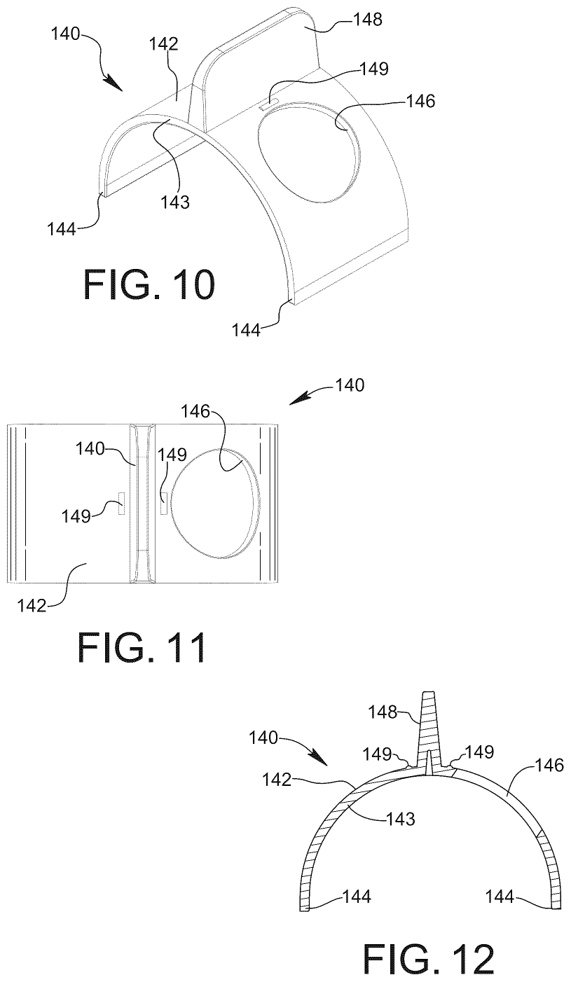

As shown in FIGS. 3-12, dust cover 100 may generally comprise a cover unit 110 and a closure member 140 coupled to the cover unit. Cover unit 110 may define an inner enclosure space 111 and a mounting opening 112 communicating with the inner enclosure space 111. The size and shape of cover unit 110 may be designed to be removably coupled to measurement head 210 via the mounting opening 112. For example, the shape and size of cover unit 110 and mounting opening 112 may be designed to allow cover unit 110 to be press-fitted onto measurement head 210 and retained on measurement head 210 by friction, such that cover unit dust cover 100 may be pulled off and removed from measurement head 210 to allow a measurement to be taken. In the illustrated example, an internal surface 114 of cover unit 110 and an external surface 214 of measurement head 210 are slightly tapered and internal surface 114 includes a plurality of angularly spaced ribs 119 to facilitate mounting and removal of dust cover 100. If eye cup 220 is present, it may be contained within the inner enclosure space 111 of the cover unit 110 to be protected from damage and contamination.

Cover unit 110 may include a funnel portion 116 that opens through an end wall 113 of the cover unit opposite from mounting opening 112. Funnel portion 116 may extend along a probe-loading axis 117. When cover unit 110 is removably coupled to measurement head 210, probe-loading axis 117 is aligned with a probe-receiving opening 213 of probe barrel 212 on measurement head 210 for loading a probe through funnel portion 116. Funnel portion 116 may have a mouth 118 and a probe-loading orifice 120. Mouth 118 may be defined at an end of cover unit 110 opposite from mounting opening 112. Probe-loading orifice 120 may be axially located between mouth 118 and mounting opening 112. Mouth 118 and probe-loading orifice 120 may be concentric with probe-loading axis 117. An area of probe-loading orifice 120 may be reduced relative to an area of mouth 118. As best seen in FIG. 8, the shape of mouth 118 may be oval and the shape of probe-loading orifice 120 may be circular in axial plan view, however other shapes are possible.

Cover unit 110 may further include a tube portion 122 extending from funnel portion 116 toward mounting opening 112. Tube portion 122 may have a probe-loading passageway 124 that is coaxial with and extends from probe-loading orifice 120. Probe-loading passageway 124 may be substantially cylindrical and may have a diameter that is less than or equal to a diameter of probe-loading orifice 120. Tube portion 122 may further have a probe-delivering orifice 126 at an opposite end of probe-loading passageway 124 relative to probe-loading orifice 120. Probe-delivering orifice 126 may be axially located between probe-loading orifice 120 and mounting opening 112.

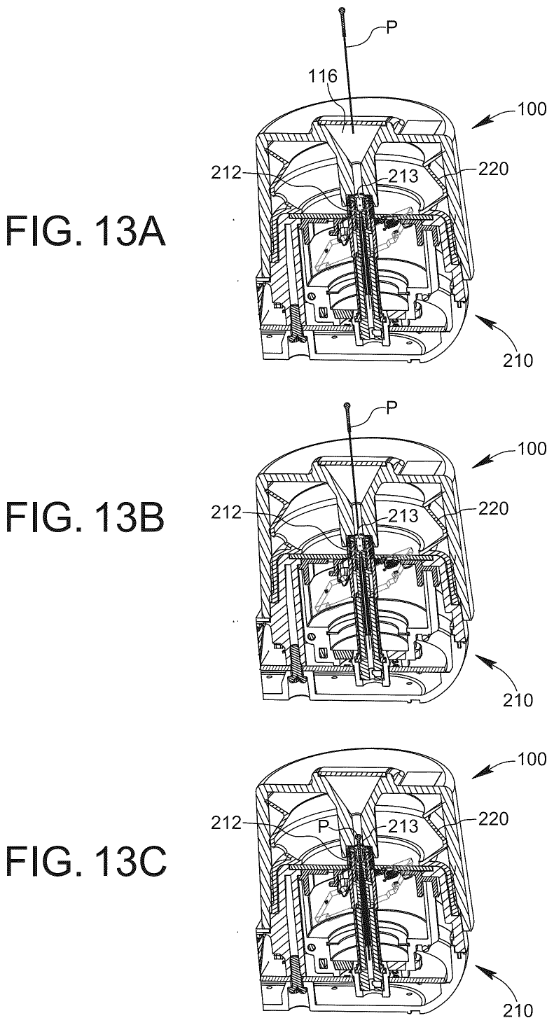

Tube portion 122 may engage with probe barrel 212 when dust cover 100 is coupled to measurement head 210 of ophthalmic instrument 200. For example, as may be seen in FIGS. 4 and 13A-13C, a diameter of probe-loading passageway 124 at region 128 adjacent probe-delivering orifice 126 may be enlarged to receive an end portion of probe barrel 212. Alternatively, tube portion 122 and barrel 212 may be configured such that an end of tube portion 122 may be received in a recess formed in the end portion of barrel 212. Under either arrangement, funnel portion 116 and tube portion 122 of cover unit 110 may provide a continuous pathway for a probe to be inserted into probe barrel 212. A probe may be inserted through funnel portion 116 via the mouth 118 and may be directed to probe-loading orifice 120 to be aligned with probe-loading axis 117 for loading into probe barrel 212 of measurement head 210.

Cover unit 110 may include a pair of slots 130 opening through end wall 113 and communicating with inner enclosure space 111. Cover unit 110 may further include an arcuate surface portion 132 between the pair of slots 130. As used herein, the terms “arcuate” and “arc” may refer to a shape defined by a portion of the boundary of a circle or a curve. Mouth 118 of the funnel portion 116 may open through arcuate surface portion 132. In one embodiment, arcuate surface portion 132 may have a radius of 16 millimeters.

Closure member 140 may be selectively movable relative to cover unit 110 between an open position and a closed position. In the open position, mouth 118 of funnel portion 116 is not fully covered by the closure member 140 to facilitate loading of a probe through mouth 118 of funnel portion 116. In the closed position, mouth 118 of funnel portion 116 is fully covered by closure member 140 to prevent dust and other contaminants from entering probe-receiving opening 213 of probe barrel 212 on measurement head 210.

The closure member 140 may include an arcuate strip 142 having a pair of opposite ends 144. The pair of opposite ends 144 of arcuate strip 142 may be received through the pair of slots 130 of cover unit 110 such that the arcuate strip 142 is in contact with arcuate surface portion 132 of the cover unit. In some embodiments, the arcuate profiles of the contacting surfaces of arcuate strip 142 and arcuate surface portion 132 may be substantially similar such that the arcuate strip 142 is flush with the arcuate surface portion 132.

Closure member 140 may further include an insertion hole 146 through arcuate strip 142. Insertion hole 146 may correspond in size and shape to mouth 118. An area of insertion hole 146 may be greater than an area of probe-loading orifice 120. The area of the insertion hole 146 may be greater than or equal to the area of mouth 118.

Closure member 140 may be slidable relative to cover unit 110 to selectively align insertion hole 146 with mouth 118 of funnel portion 116 in an open position. For example, a center of insertion hole 146 may be aligned with probe-loading axis 117 in a fully open position. In some embodiments, a boundary of mouth 118 may be located within, or may be coincident with, a boundary of insertion hole 123 in the fully open position. Closure member 140 may be slidable relative to the cover unit 110 to cover mouth 118 of funnel portion 116 with arcuate strip 142 in a closed position. Accordingly, by sliding the closure member 140 relative to the cover unit 110, mouth 118 can be selectively covered by arcuate strip 142 to protect probe-receiving opening 213 of probe barrel 212 from dust and contaminants, and mouth 118 can be selectively opened fully or partially to expose at least a portion of mouth 118 through insertion hole 123 to receive a probe in an open position.

Closure member 140 may include a tab 148 protruding from arcuate strip 142. In some embodiments, tab 148 may protrude from arcuate strip 142 at a location midway between the pair of opposite ends 144 of arcuate strip 142. Insertion hole 146 may be positioned on arcuate strip 142 at a location between tab 148 and one end 144 of arcuate strip 142. Tab 148 may be configured for engagement by a user to manually move closure member 140 relative to cover unit 110. For example, a user may press or grip tab 148 to selectively move closure member 140 between an open position and a closed position. In some embodiments, tab 148 may abut against cover unit 110 (e.g., against an edge of one slot 130) in the open position to limit further slidable movement of the closure member 140 relative to the cover unit 110 and align the insertion hole 146 with mouth 118. Tab 148 may also abut against the cover unit 110 (e.g., against an edge of the other slot 130) in a closed position to limit further slidable movement of the closure member 140 relative to the cover unit 110 and cover the mouth 118 with arcuate strip 142. Accordingly, a user can easily toggle the closure member 110 back and forth between an open position and a closed position based on the physical limits of the slidable movement of the closure member 140 established by the tab 148. Closure member 140 may include a pair of raised bumps 149 on its outer surface respectively positioned on opposite sides of tab 148 for engaging with an edge of a corresponding slot 130 to provide a tactile and/or audible indication to a user that closure member 140 has reached an open position or a closed position relative to cover unit 110.

Arcuate strip 142 may include a circular arc portion 143 bounded by a pair of opposite end portions 144 which may extend in a tangential direction from opposite ends of circular arc portion 143. As shown by example in the drawings, circular arc portion 143 may measure 180 degrees. In one embodiment, a radius of an inner surface of circular arc region 143 may be 16.25 millimeters, and opposite end portions 144 may extend 1.7 millimeters from the opposite ends of circular arc portion 143, respectively. Arcuate strip 142 may be dimensioned such that the arcuate strip must be resiliently flexed, e.g., by bringing opposite end portions 144 slightly closer together to enable the end portions 144 to be received through the pair of slots 130 in cover unit 110, whereby arcuate strip is spring-biased to exert a lateral force against the outer edges of slots 130. Advantageously, the lateral force introduces frictional resistance to sliding movement of closure member 140 relative to cover unit 110, which may prevent accidental movement of closure member 140 between open and closed positions. The lateral force may retain closure member 140 in an open position when a user is loading a probe and may retain closure member 140 in a closed position when ophthalmic instrument 200 is not in use.

FIGS. 13A through 13C illustrate a process of loading a probe P into the ophthalmic instrument 200 using the dust cover 100. For clarity, closure member 140 is not shown, but it may be understood that closure member 140 is in an open position during the process. In FIG. 13A, the probe P is being placed into funnel portion 116 of cover unit 110 by way of mouth 118. In FIG. 13B, a leading end of probe P is moving down probe-loading passageway 124 of tube portion 122 of the cover unit 110 after passing through probe-loading orifice 120. In FIG. 13C, the leading end of probe P has passed through probe-delivering orifice 126 and has entered probe-receiving opening 213 of probe barrel 212 on measurement head 210. Accordingly, probe P may arrive at a position suitable for use in a measurement by ophthalmic instrument 200. After loading probe P, the user may remove dust cover 100 from measurement head 210 of ophthalmic instrument 200 to proceed with a measurement, for example by pulling dust cover 100 axially away from measurement head 210. In this way, dust cover 100 may be uncoupled from ophthalmic instrument 200 without risk of damaging probe P, which may be safely positioned within the barrel 212.

After measurement, probe P may be removed from probe barrel 212 and dust cover 100 may once again be coupled to measurement head 210 with closure member 140 moved to a closed position for conveniently and safely storing ophthalmic instrument 200. As a result, probe barrel 212 and probe-receiving opening 213 may be protected from dust and other contaminants without the use of a separate storage case for ophthalmic instrument 200.

While the present disclosure describes exemplary embodiments, the detailed description is not intended to limit the scope of the appended claims to the particular embodiments set forth. The claims are intended to cover such alternatives, modifications and equivalents of the described embodiments as may be included within the scope of the claims.

What is claimed is:

1. An apparatus comprising:
a cover unit defining an inner enclosure space and a mounting opening communicating with the inner enclosure space; and
a closure member coupled to the cover unit;
wherein the cover unit includes a funnel portion extending along a probe-loading axis, the funnel portion having a mouth and a probe-loading orifice, wherein an area of the probe-loading orifice is reduced relative to an area of the mouth;
wherein the closure member is selectively movable relative to the cover unit between an open position in which the mouth of the funnel portion is not fully covered by the closure member and a closed position in which the mouth of the funnel portion is fully covered by the closure member.

2. The apparatus according to claim 1, wherein the cover unit includes a pair of slots and an arcuate surface portion between the pair of slots, the mouth of the funnel portion opens through the arcuate surface portion, and the closure member includes an arcuate strip having a pair of opposite ends and an insertion hole through the arcuate strip, wherein the pair of opposite ends of the arcuate strip are received through the pair of slots.

3. The apparatus according to claim 2, wherein a center of the insertion hole is aligned with a center of the mouth of the funnel portion when the arcuate strip is in the open position.

4. The apparatus according to claim 2, wherein the closure member further includes a tab protruding from the arcuate strip for engagement by a user to move the closure member relative to the cover unit.

5. The apparatus according to claim 4, wherein the tab protrudes from the arcuate strip at a location midway between the pair of opposite ends of the arcuate strip.

6. The apparatus according to claim 4, wherein the insertion hole is positioned on the arcuate strip between the tab and one end of the pair of opposite ends.

7. The apparatus according to claim 2, wherein an area of the insertion hole is greater than the area of the probe-loading orifice.

8. The apparatus according to claim 7, wherein the area of the insertion hole is greater than or equal to the area of the mouth.

9. The apparatus according to claim 4, wherein the closure member includes a pair of raised bumps respectively positioned on opposite sides of the tab.

10. The apparatus according to claim 2, wherein the arcuate strip includes a circular arc portion.

11. The apparatus according to claim 10, wherein the circular arc portion measures 180 degrees.

12. The apparatus of claim 4, wherein the arcuate strip is dimensioned such that the arcuate strip is flexed when the pair of opposite ends of the arcuate strip are received through the pair of slots, whereby the arcuate strip is spring-biased to exert a lateral force against respective edges of the pair of slots.

13. The apparatus according to claim 1, wherein the cover unit further includes a tube portion extending from the funnel portion toward the mounting opening, wherein the tube portion has a probe-loading passageway coaxial with and extending from the probe-loading orifice.

14. The apparatus according to claim 13, wherein the tube portion has a probe-delivering orifice at an end of the probe-loading passageway opposite the probe-loading orifice.

15. The apparatus according to claim 14, wherein the probe-delivering orifice is axially located between the probe-loading orifice and the mounting opening.

16. The apparatus according to claim 1, wherein the probe-loading orifice is axially located between the mouth of the funnel portion and the mounting opening.

17. The apparatus according to claim 1, further comprising an ophthalmic instrument having a measurement head including a probe barrel having a probe-receiving opening, wherein the cover unit and closure member are removably mountable on the measurement head.
